(19) United States
(12) Patent Application Publication
(10) Pub. No.: US 2024/0269002 A1
(43) Pub. Date: Aug. 15, 2024

(54) METHOD FOR PROVIDING CONTROL DATA FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/436,890

(22) Filed: Feb. 8, 2024

(30) Foreign Application Priority Data
Feb. 10, 2023 (DE) 10 2023 103 285.2

(51) Int. Cl. A61F 9/008

(52) U.S. Cl. CPC A61F 9/00804; A61F 2009/00872

(57) ABSTRACT

The techniques presented herein relate to ascertaining a pretherapeutic curvature of the cornea, wherein a respective local angle of the cornea in predetermined treatment positions is ascertained; ascertaining a radiation angle at which a laser beam is radiated to the respective predetermined treatment positions of the cornea with respect to a reference axis of a beam deflection device of the treatment apparatus; and ascertaining a laser pulse efficiency for a particular treatment position of the ascertained predetermined treatment positions based on the ascertained local angle of the cornea in the particular treatment position and the radiation angle for the particular treatment position. The techniques further include adapting at least one irradiation parameter depending on the ascertained laser pulse efficiency for the particular treatment position; and providing the control data, which includes adapted irradiation parameters for the particular treatment position.

### FIELD

The invention relates to a method for providing control data for an ophthalmological laser of a treatment apparatus for the treatment of a cornea of an eye. Furthermore, the invention relates to a control device, which is configured to perform the method, to a treatment apparatus with such a control device, to a computer program and to a computer-readable medium, on which the computer program is stored.

### BACKGROUND

Treatment apparatuses and methods for controlling lasers for correcting an optical visual disorder of a cornea are known in the prior art. Therein, a pulsed laser and a beam deflection device or beam focusing device can for example be formed such that laser beam pulses effect a photodisruption or ablation in a focus situated within the tissue of the cornea to separate a lenticule from the cornea for correcting the cornea.

Available methods for planning control data allow a correction of refractive visual disorders such as for example myopia, hyperopia or astigmatism. However, a spherical aberration is often unintentionally induced by a treatment, by which halos and a reduced contrast sensitivity are generated. These undesired effects often arise due to a varying laser pulse efficiency between different treatment positions, in particular in radial direction. Herein, it is often difficult to keep the laser pulse efficiency constant for each treatment position to thus achieve the planned treatment result for each treatment position.

Therefore, it is the object of the invention to be able to provide a laser pulse energy for a respective treatment position in an improved manner.

### SUMMARY

This object is solved by the independent claims. Advantageous embodiments are disclosed in the dependent claims, the following description as well as the figures.

The invention is based on the idea that a deviation of the laser pulse efficiency arises in that laser pulses impinge on the cornea to be treated at different angles in particular in radial direction. Accordingly, it is provided that the laser pulse efficiency is adapted depending on an angle of incidence on the cornea to preferably obtain a constant laser pulse efficiency.

An aspect of the invention relates to a method for providing control data for an ophthalmological laser of a treatment apparatus for treating a cornea of an eye, wherein the following steps are performed by a control device. Ascertaining a pretherapeutic curvature of the cornea is effected, wherein a respective local angle of the cornea in respective predetermined treatment positions is ascertained. This means that a local curvature or a local inclination is determined in each position of the cornea, which is intended for the treatment. This can for example be an average curvature of the cornea, in particular considering astigmatism and asphericity. Herein, examination data can for example be preset, in particular a topography, from which the curvature of the cornea can be determined for preset treatment positions before the treatment.

Furthermore, ascertaining a radiation angle, at which a laser beam is radiated to the respective predetermined treatment positions of the cornea with respect to a reference axis, in particular a neutral axis, of a beam deflection device of the treatment apparatus is effected. This means that a radiation angle of the laser beam can for example be ascertained by means of a treatment position, which is located in radial direction of the cornea. The radiation angle is preferably defined in relation to a reference axis, in particular a neutral axis or to a neutral position, of the beam deflection device. The reference axis can have a preset reference angle, at which the beam deflection device radiates the laser beam to the cornea. In particular, the reference angle can be determined based on the neutral axis of the beam deflection device, whereby the radiation angle presents an offset to a neutral position of the neutral axis. The beam deflection device can be formed as a scanner in x-, y- and/or z-direction, in particular as a rotation scanner. The laser beam, which can be radiated by the ophthalmological laser and deflected by the beam deflection device to the respective treatment positions, can preferably include multiple laser pulses, which generate an optical breakthrough on or in the cornea.

Furthermore, the method includes ascertaining a laser pulse efficiency of the respective treatment position depending on the ascertained local angle of the cornea in the treatment position and the radiation angle for the treatment position and adapting at least one irradiation parameter depending on the ascertained laser pulse efficiency for the respective treatment position. This means that the prospective laser pulse efficiency is determined for each treatment position in that an angle of incidence of the laser beam on the respective treatment position is determined, wherein the angle of incidence can be calculated from the ascertained local angle and the radiation angle of the laser beam. Based on the angle of incidence, the laser pulse efficiency for the respective treatment position can then be determined by means of a (mathematical/physical) model and/or predetermined measurements. The respective irradiation parameter can then be adapted for the corresponding treatment position depending on the determined laser pulse efficiency to compensate for this effect, wherein the adaptation is preferably effected with a reciprocal value of the laser pulse efficiency such that the laser pulse efficiency is kept constant. For example, a laser pulse energy and/or a number of laser pulses in the corresponding treatment position can for example be adapted as the irradiation parameter.

Finally, providing the control data, which includes adapted irradiation parameters for the respective treatment position, is effected. Preferably, the treatment apparatus, in particular the laser, can then be controlled with the control data for correcting a visual disorder.

In other words, visual disorder correction data with respective treatment positions can be predetermined for generating a correction profile in the method, wherein at least one irradiation parameter of the treatment apparatus is adapted depending on an irradiation position in or on the cornea by means of the method, to preferably obtain the same laser pulse efficiency in each treatment position. Therein, the laser pulse efficiency is depending on the fact, with which steepness the pulse impinges on the cornea in the respective treatment position, since the laser pulse efficiency is composed of reflection losses, geometric distortions and superpositions of adjacent pulses. In particular the steeper or more inclined a pulse impinges on the cornea, the larger is a surface, to which the energy distributes, and a higher portion of reflection losses arises. In order to determine this angle of incidence on the cornea, the local angle of the cornea in the treatment position on the one hand and the radiation angle of the laser beam on the other hand are determined. Then, the steepness or the angle of incidence, at which the laser pulse impinges on the cornea, and thus the laser pulse efficiency, can then be determined from these two angles by means of geometric calculation.

Therein, the laser pulse efficiency for the respective treatment position can be calculated from the physical model. Alternatively or additionally, a respective laser pulse efficiency for respective angles of incidence can be predetermined from measurements. The control data can include a respective dataset for positioning and/or for focusing individual laser pulses in the cornea. In addition, a respective dataset for adjusting an irradiation parameter can be included in the control data, in particular of a beam device for beam guidance and/or beam shaping and/or beam deflection and/or beam focusing of a laser beam of the respective laser.

By the invention, the advantage arises that effects, which occur by different angles of incidence on the treatment positions, can be avoided or reduced, whereby an improved treatment result arises. In particular, spherical aberrations can be minimized.

The invention also includes embodiments, by which additional advantages arise.

In an embodiment a laser pulse energy is adapted as a function of the treatment position as the irradiation parameter. Herein, the angle of the local curvature can be predetermined for a respective treatment position and the radiation angle for the respective treatment position can be ascertained. Thus, the laser pulse energy can be increased or reduced to compensate for an effect of the angle of incidence. For example, the laser pulse energy can be adjusted depending on a radial position of the cornea, by which the treatment position can be provided. By this embodiment, an advantageous configuration for adapting the laser pulse efficiency can be provided.

In a further embodiment a number of laser pulses of the laser is adapted as a function of the treatment position as the irradiation parameter. This means that a repetition rate of the laser can for example be adapted, wherein the repetition rate can in particular be increased with increasing steepness of the curvature. Alternatively, the laser pulse energy can preferably be kept constant and only the number of the pulses, which is irradiated to the respective treatment position, is adapted. This means that the repetition rate of the laser is preferably not changed, but a duration, with which a treatment position is irradiated by the laser beam, is adapted, to thus increase the number of the pulses onto the treatment position. Hereby, a further preferred configuration for adaptation of the laser pulse efficiency can be achieved.

In a further embodiment a laser pulse distance of adjacent laser pulses is adapted as a function of the treatment position as the irradiation parameter. In other words, the beam deflection device or the scanner can move slower or faster according to treatment position to adjust the laser pulse distance of adjacent laser pulses. Thus, a spot distance of the laser pulses can be reduced or increased, which affects the energy density. Herein, the advantage arises that an adjustment via the beam deflection device can be easier performed than a change of the laser pulse energy and the repetition rate, respectively, of the laser.

In a further embodiment the pretherapeutic curvature of the cornea is ascertained from a topography of the cornea. This means that examination data can be provided, which includes a topography measurement of the cornea, wherein a topography can be established from the examination data, by means of which the curvature in each treatment position can be analytically or numerically ascertained.

In a further embodiment the pretherapeutic curvature is ascertained based on a preset curvature of a contact element. Herein, a contact element can be a patient interface, onto which the eye or the cornea can be pressed for the treatment to for example retain the eye and/or to bring the eye into a preset curvature. Accordingly, by the curvature of the contact element, thus, the curvature of the cornea and the respective local angles are also known. Hereby, the advantage arises that further calculations for determining the pretherapeutic curvature are not required, but it is known for each treatment position from the preset curvature of the contact element, which simplifies the method.

In a further embodiment a change of the curvature of the cornea by treatment with the laser pulses is additionally ascertained, from which a changed angle of the respective still unirradiated treatment position is determined, wherein the changed angle is used in ascertaining the laser pulse efficiency. In other words, a change of the curvature can occur during the treatment, for example flattening or tapering. It can be planned in advance by a suitable laser pulse pattern, this means that it can be known when which treatment position is irradiated and to which extent the curvature has then changed at the point of time of the irradiation in a respective treatment position. Thus, one knows to what extent the angle has changed up to the point of time, at which the treatment position is irradiated, wherein this changed angle is then taken into account for determining the laser pulse efficiency. Thus, one obtains a progressive effect. In particular, the change of the curvature can be determined for subsequent pulses in that the local curvature in subsequent treatment positions is ascertained in statistical manner, for example after half of the pulses, or in iterative manner, for example after a predetermined number of pulses. By this embodiment, the advantage arises that a laser pulse efficiency can be even better adapted to a current local curvature and thus an angle of incidence, which overall improves the treatment.

In a further embodiment an eye orientation is tracked by a capturing device during the treatment, wherein the eye orientation is involved in the adaptation of the irradiation parameter for subsequent treatment positions. For example, the capturing device can include one or more cameras, which ascertain a viewing direction of the eye. This means that the capturing device can be an eye tracking system, whereby the eye orientation is known at each point of time of the treatment. Upon change of the eye orientation, thus, the treatment position and thus the radiation angle and/or the local curvature can also change, wherein this can be compensated for based on the ascertained eye orientation. Hereby, the advantage arises that a 3D rotation of the eyeball can be tracked, whereby an improved adaptation of the irradiation parameters and thus of the laser pulse efficiency can be achieved.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate a predefined corneal volume with predefined interfaces of a human or animal eye by means of optical breakthrough, in particular at least partially separate it by means of photodisruption and/or to ablate corneal layers by means of (photo)ablation and/or to effect a laser induced refractive index change in the cornea and/or the eye lens and/or to increase a crosslinking of the cornea.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims.

FIG. 1 depicts a schematic representation of a treatment apparatus according to an exemplary embodiment.

FIG. 2 depicts a flow diagram of a method for providing control data according to an exemplary embodiment.

In the figures, identical or functionally identical elements are provided with the same reference characters.

### DETAILED DESCRIPTION

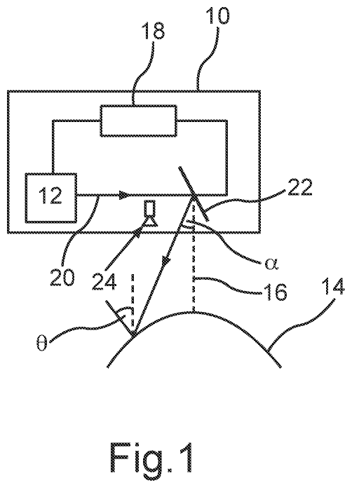

The FIG. 1 shows a schematic representation of a treatment apparatus 10 with an eye surgical laser 12 for the correction of a cornea 14, wherein a correction profile, in particular a volume body or a lenticule (not shown), can for example be defined by control data, which can be separated from the cornea 14 by means of photodisruption and/or ablation. For separating the lenticule, interfaces can for example be preset in the control data, on which a cavitation bubble path for separating the lenticule from the cornea 14 can be generated.

A control device 18 for the laser 12 can be formed besides the laser 12, such that it can emit pulsed laser pulses, for example, in a predefined pattern for generating the correction profile or the interfaces. Alternatively, the control device 18 can be a control device 18 external with respect to the treatment apparatus 10.

Furthermore, the FIG. 1 shows that the laser beam 20 generated by the laser 12 is deflected towards the cornea 14 by means of a beam deflection device 22 such as, for example, a rotation scanner. The beam deflection device 22 is also controlled by the control device 18 to generate the correction profile or the interfaces. Herein, the beam deflection device 22 can radiate the laser beam 20 to the cornea 14 on a neutral axis 16 in a neutral position, wherein further treatment positions of the cornea 14, which can for example be defined by means of coordinates in x- and y-direction or a radial direction, can be achieved by adaptation of a radiation angle α. Herein, the radiation angle α can be defined as the angle between the radiation direction to the treatment position and the neutral axis 16, wherein a radiation in the direction of the neutral axis 16 can preferably be defined as a radiation angle or reference angle of 0°.

Preferably, the illustrated laser 12 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. In addition, the control device 18 optionally comprises a storage device (not illustrated) for at least temporary storage of at least one control dataset, wherein the control dataset or datasets include(s) control data for positioning and/or for focusing individual laser pulses in the cornea. The position data and/or the focusing data of the individual laser pulses, that is the correction profile of the lenticule to be separated, are generated based on predetermined control data, in particular from previously measured visual disorder data, in particular a previously measured topography and/or pachymetry and/or the morphology of the cornea.

According to the fact, at which radiation angle α the laser beam 20 is radiated to the cornea 14 by the beam deflection device 22, geometric conditions of an angle of incidence of the laser beam 20 to the cornea 14 can change, whereby the laser pulse efficiency can also change. Thus, the laser pulse efficiency can for example be maximum upon perpendicular impingement on the cornea 14, for example along the neutral axis 16, and decrease with increasing radiation angle. By a change of the angle of incidence on the cornea 14, the laser pulse efficiency of the respective laser pulses on the treatment position can in particular be composed due to a changed reflection characteristic, geometric distortions and superpositions of adjacent pulses.

Furthermore, the angle of incidence of the laser beam on the cornea 14 and thus the laser pulse efficiency can also depend on a local curvature of the cornea 14 besides the radiation angle α. The curvature of the cornea 14 can be provided by a local angle θ in each treatment position, which is preferably defined as an angle between the neutral axis 16 and a normal vector on the respective treatment position.

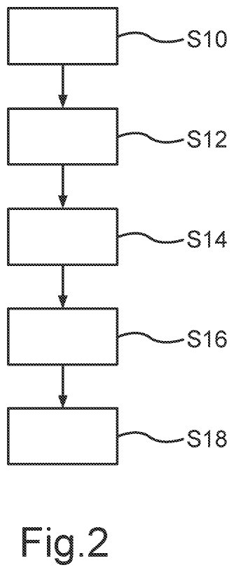

In order to consider the effects of the variable laser pulse efficiency depending on the angle of incidence of the laser on the cornea 14 in the treatment, a method for providing control data schematically illustrated in FIG. 2 can be performed.

In a step S10, a pretherapeutic curvature of the cornea 14 can be ascertained, wherein a local angle θ of the cornea 14 is determined in respective predetermined treatment positions. The pretherapeutic curvature of the cornea 14 can for example be ascertained by means of a previously performed topography measurement. Alternatively or additionally, the cornea 14 can be pressed onto a contact element (not shown) for the treatment, wherein a curvature of the contact element is preferably known. Thus, the angle θ for the respective treatment position can be determined from the preset curvature of the contact element.

In a step S12, it can be determined, which radiation angle α is planned for a respective treatment position, wherein the radiation angle α can preferably be defined with respect to a neutral axis of the beam deflection device 22, the reference angle of which is for example set as 0 degrees. Thus, an offset to a neutral position of the beam deflection device 22, which can be preset by the neutral axis 16, can be determined to obtain the radiation angle α.

In a step S14, a laser pulse efficiency can then be ascertained for the respective treatment position depending on the ascertained local angle θ and the ascertained radiation angle α. Herein, the laser pulse efficiency can be predetermined for respective angles of incidence, which result from the local angle θ and the radiation angle α, for example by means of preceding measurements. Alternatively, the laser pulse efficiency can be calculated by means of a mathematical or physical model. Thus, a change of the laser pulse efficiency with respect to the neutral axis can for example be determined from a change of a respective surface by the changed angle of incidence, in particular by means of geometric considerations, and/or a change of reflection characteristics can be calculated by change of the angle of incidence and thus of reflection losses. Furthermore, overlap effects and losses, which occur due to an attenuation upon passage through the cornea 14, can also be taken into account, in particular by means of the Lambert-Beer law.

In a step S16, at least one irradiation parameter can then be adapted depending on the ascertained laser pulse efficiency for the respective treatment position, wherein the adaptation is preferably performed with a reciprocal value of the laser pulse efficiency to keep the laser pulse efficiency constant for each treatment position. As the irradiation parameter, a laser pulse energy and/or a number of pulses on a treatment position can in particular be adapted. For example, a laser pulse energy and a repetition rate, respectively, can be increased to adapt a loss of a laser pulse efficiency depending on the treatment position. Preferably, it is provided that the laser pulse energy and/or the repetition rate are kept constant and only the number of the pulses for a treatment position is increased in that a longer irradiation duration for a respective treatment position and/or a lower laser pulse distance are for example adjusted by adaptation of the beam deflection device 22.

Preferably, the eye or the cornea 14 can also be monitored by a capturing device 24 during the treatment, which can comprise one or more cameras, and which is formed to determine an eye orientation. By means of the eye orientation, which can provide a 3D rotation of the eyeball, thus, a change of the radiation angle α and/or of the local angle θ can be ascertained, which can preferably be taken into account in the adaptation of the laser pulse efficiency for the respective treatment position.

Furthermore, it can be ascertained or predetermined, which change of the curvature of the cornea 14 is expected by the treatment, wherein an angle changed thereby to respective still unirradiated treatment positions can be determined, which can be used for determining the laser pulse efficiency. Herein, a redetermination of the curvature of the cornea 14 and thus of the local angle θ can for example be performed after a preset number of pulses to perform an adaptation of the ascertainment of the laser pulse efficiency.

Finally, control data for the ophthalmological laser 12 of the treatment apparatus 10 can be provided in a step S18, in which adapted irradiation parameters are provided for each treatment position. The laser 12 and/or the beam deflection device 22 can then be controlled by means of the control data for treating the cornea 14.

Overall, the examples show how a geometric loss of a laser pulse efficiency can be compensated for by the invention.

1. A method, performed by a control device, for providing control data for an ophthalmological laser of a treatment apparatus for treating a cornea of an eye, wherein the method comprises:
ascertaining a pretherapeutic curvature of the cornea, wherein a respective local angle of the cornea in predetermined treatment positions is ascertained;
ascertaining a radiation angle at which a laser beam is radiated to the respective predetermined treatment positions of the cornea with respect to a reference axis of a beam deflection device of the treatment apparatus;
ascertaining a laser pulse efficiency for a particular treatment position of the ascertained predetermined treatment positions based on the ascertained local angle of the cornea in the particular treatment position and the radiation angle for the particular treatment position;
adapting at least one irradiation parameter depending on the ascertained laser pulse efficiency for the particular treatment position; and
providing the control data, which includes adapted irradiation parameters for the particular treatment position.

2. The method according to claim 1, wherein the at least one irradiation parameter includes a laser pulse energy.

3. The method according to claim 1, wherein the at least one irradiation parameter includes a number of laser pulses of the ophthalmological laser.

4. The method according to claim 1, wherein the at least one irradiation parameter includes a laser pulse distance of adjacent laser pulses.

5. The method according to claim 1, wherein the pretherapeutic curvature of the cornea is ascertained from a topography of the cornea.

6. The method according to claim 1, wherein the pretherapeutic curvature is ascertained based on a preset curvature of a contact element.

7. The method according to claim 1, further comprising:
ascertaining a change of curvature of the cornea resulting from treatment with the laser,
determining a changed angle of a respective unirradiated treatment position, and
further ascertaining the laser pulse efficiency based on the changed angle.

8. The method according to claim 1, wherein an eye orientation is tracked by a capturing device during the treatment, wherein the the at least one irradiation parameter is further adapted for subsequent treatment positions based on the eye orientation.

9. A method for controlling a treatment apparatus, wherein the method comprises:
the method according to claim 1, and
transferring the provided control data to the ophthalmological laser of the treatment apparatus.

10. A control device, configured to perform the method according to claim 1.

11. A treatment apparatus with at least one ophthalmological laser for separation of a corneal volume with predefined interfaces of a human or animal eye by means of optical breakthrough, photodisruption and/or photoablation, and the at least one control device according to claim 10.

12. (canceled)

13. A non-transitory computer-readable medium, on which a computer program is stored, the computer program comprising commands which, when executed by the control device, cause the treatment apparatus, with at least one ophthalmological laser for separation of a corneal volume with predefined interfaces of the eye, to perform the method according to claim 1.
